(19) United States
(12) Patent Application Publication
(10) Pub. No.: US 2013/0344454 A1
(43) Pub. Date: Dec. 26, 2013

(54) Optical Irradiation Appliance for Dermatology and Beauty Care

(71) Applicant: Günther Nath, Grunwald (DE)

(72) Inventor: Günther Nath, Grunwald (DE)

(21) Appl. No.: 13/884,557

(22) PCT Filed: Nov. 10, 2011
(86) PCT No.: PCT/EP2011/069888

§ 371 Date: Jul. 29, 2013

(30) Foreign Application Priority Data
Nov. 10, 2010 (EP) PCT/EP2010/067192
Jan. 17, 2011 (DE) 10 2011 008 705.2
Mar. 15, 2011 (DE) 10 2011 013 988.5

(51) Int. Cl. A61N 5/06

(52) U.S. Cl. CPC A61N 5/0613
USPC 433/29; 607/90

(57) ABSTRACT

The appliance according to the invention for dermatological or cosmetic treatment of a patient comprises a base body (19; 6c19; 59), which has a radiation source (11; 6c11; 21; 41; 61) for emitting light in a wavelength range between 350 nm and 1000 nm. An intermediate piece (15; 6c15, 6c22, 6c23, 6c25; 25; 35; 45; 55; 550, 551; 650) sitting on the base body (19; 6c19; 59) conveys the light from the radiation source (11; 6c11; 21; 41; 61) in the direction of a body region (214) of the patient to be treated. An attachment element (15; 6c15, 6c22, 6c23, 6c25; 25; 35; 45; 55; 550, 551; 650) secured detachably on the light output end of the intermediate piece (15; 6c15, 6c22, 6c23, 6c25; 25; 35; 45; 55; 550, 551; 650) is brought into direct contact with the body region (214) to be treated, in order thereby to introduce a dermatologically or cosmetically active substance (213), during irradiation with the emitted light, into the body region (214) to be treated. Alternatively, the attachment element (540) can also simply serve to adapt the radiation cross-section of the emitted light to the size and the irregularities of the body region to be treated.

### TECHNICAL FIELD

The present invention concerns improvements to an appliance for dermatological or cosmetic treatment of a patient. In particular, the effectiveness of dermatological or cosmetic substances for application to the human skin is to be enhanced by optical and mechanical aids.

### PRIOR ART

The present invention constitutes a further development of my dermatological irradiation appliance as described in PCT/EP2010/067192. The focus of the previous PCT application is in the field of treatment of nail fungus by the joint action of optical radiation and a peroxide-containing paste. As a result of optically induced radical formation, a softening of fungus-infiltrated nail substance occurs within a few minutes. This effect is helpful in eliminating the fungus-attacked nail material.

### DESCRIPTION OF THE INVENTION

The problem of the present invention is to broaden the area of application of the technique described in PCT/EP2010/067192 to other indications of dermatology and beauty care and to indicate corresponding technical adaptations.

In particular, the problem is to introduce active substances which are contained in dermatological salves, gels or pastes more quickly and deeply into the uppermost layers of skin and thus accomplish a better action by means of intense light. As an example, one can mention here the introducing of anti-inflammatory or painkilling gels, salves or pastes into the skin with effective support of intense optical radiation while at the same time applying pressure to an area of tissue.

Voltaren® pain gel or Aloe Vera Gel® or heparin-containing gels are only three specific examples of many dermatological gels, salves or tinctures that are well suited to optical introduction into the skin.

Another application of the irradiation appliance of the present invention lies in the field of beauty care. This involves first and foremost the improved smoothing of wrinkles in the skin by combined action of optical radiation and conventional cosmetic gels, salves or liquids and application of pressure to an area of tissue.

As an example, we mention the better and faster introduction of Hyaluron®-containing gels, salves or liquids into the skin by means of intense optical radiation and application of pressure to an area of tissue.

Specifically, the irradiation appliance of the invention should meet the following requirements:

1) It should be small and light and able to be operated by hand.

2) Despite its small size and easy handling, it should generate a high radiation power density of at least 50 to 100 mW/cm² on an area of tissue of around 0.5 to 3 cm in diameter, so that after at most 30 seconds one can already feel the effect of the heat on the skin.

3) The irradiation appliance should generally be able to make contact with the area being irradiated via a tissue pressure element (hereinafter also called the attachment element or cap) so as to always create a reproducibly identical radiation power density at the tissue.

4) The tissue pressure element must be highly transparent. Furthermore, it should be so soft and flexible as to adapt to the irregularities of the tissue when slightly pressed against it.

5) The tissue pressure element must be quickly and easily interchangeable or removable and should be medically approved for the intended use. Furthermore, it should be chemically inert to the medical and cosmetic gels, salves and liquids and be capable of being disinfected with the customary medical or cosmetic disinfecting liquids (such as Cidex®, Mucocid®, Gigasept®, and others). Even better: the tissue pressure element should be autoclavable. It should also be dishwasher-safe.

6) The tissue pressure element should have a device enabling extremely economical and leakage-free use and application of customary dermatological and/or cosmetic gels and salves. Thus, for example, a gel containing Hyaluron®, which is used in beauty care for smoothing of wrinkles, already costs 1 Euro per ml, and what is more this involves a lower concentration of active ingredient and a not particularly expensive gel.

7) The irradiation appliance should be usable without danger on the face and also in the region around the eyes, without stray radiation getting into the eye and causing a glare or damage there.

These problems are solved at least in part by the treatment appliance defined in claim 1. The subclaims 2 to 37 concern preferred sample embodiments.

According to another aspect of the present invention that is especially suited to dermatological and forensic investigations, an irradiation appliance is to be created such that wavelength and intensity of the emitted light can be optimized for special optical investigations. This problem is solved by the appliance of claim 38.

According to another aspect of the present invention that is especially suited to dental bleaching, an irradiation appliance is to be created which adapts the optical radiation cross section of the emitted light optimally to the region of the body being treated. This problem is solved by the appliance according to claim 39.

According to one sample embodiment of the invention, problems such as the following are solved:

The optical irradiation appliance contains as its light source one or more (an array of) light-emitting diodes in closest possible spatial arrangement to create maximum luminous density. A square arrangement of four individual diodes or a rectangular arrangement of 6 individual diodes has proven to work well. In total, up to 12 individual diodes can be present in approximately circular arrangement on a surface of at most 9 to 10 mm in diameter. The spectral range in which the LEDs emit can lie in the interval of 350 mm<λ<1000 nm. The electrical power consumption of the diode array can be between 5 W and 40 W.

The diode array is joined to a metallic cooling element with good thermal conductance, which at the same time can be fashioned into an elongated handle or be contained in an elongated handle. The handle has one (or more) air entry and exit openings. A fan with a cooling power of around 80 l/min carries away the heat of the cooling element.

A preferred configuration is an LED array with four single diodes in series connection in square or star arrangement with an electrical power consumption of around 10 to 15 W (15 V, 1 A).

In this configuration of the irradiation appliance, as shown in FIG. 1, continuous duty operation is possible. The handle of aluminum will get warm, but the temperature rise is still in the tolerable range and the emission power of the four LEDs also remains constant in continuous duty. With optimization of the cooling of the LEDs and the handle, the upper limit on the usable electrical power consumption of the diode array is around 30 W, provided one does not wish to sacrifice the small size and easy handling of the irradiation appliance.

The radiation of the LED array is emitted perpendicular to the axis of the handle, while a funnel-shaped reflector and also a collecting lens or a planar surface located inside a tube focus the radiation and enclose the LED array on the outside and protect against contamination.

The tissue pressure element usually in the form of a cap with tight seat is fitted onto the tube, while the cap with its radiation exit surface consists of a plastic polymer, being at least translucent or preferably highly transparent. As the material for the cap or the tissue pressure surface one can consider first and foremost silicone rubber, but also materials like fluorocarbon polymers (Teflon® FEP, Teflon® MFA, Teflon® PTFE, Hyflon® THV) or polyurethane or polyethylene (also cross-linked).

Preferable are elastomeric plastics, which are very soft and highly transparent, largely chemically inert and high-temperature resistant (>150° C.). Among these, cylindrical addition-cross-linked elastomeric silicone-rubber materials, so-called two-component silicone rubber materials, two-component polymethyl silicones with platinum catalyst, such as Elastosil® RT 601 A/B, are preferred as materials for the tissue pressure element.

The tissue pressure element generally has the shape of a cap (see FIG. 1, reference 18) and consists, e.g., of a cast two-component silicone rubber whose Shore A hardness is around 45 and whose elongation at breaking is around 100%. The tissue pressure element, e.g., in the shape of a homogeneously shaped cap (FIG. 1, 18), preferably has the attribute that one can adjust two different shaped geometries of the radiation exit or tissue pressure surface (FIG. 1, 18a), and this in reversible manner.

One geometry is concave and serves to hold a gel or a salve. With this geometry, one can spread the gel (salve) economically on the tissue surface. The second geometry is planar or slightly convex. With this geometry, the gel (salve) with the active ingredient can be massaged or introduced into the tissue upon simultaneous irradiation of the tissue with intense light.

Since a preferred light radiation used is in the spectral interval between 445 nm and 487 nm, producing an exceptionally high power density of around 400 mW/cm² at the tissue, there is an almost instantaneous and noticeable temperature rise in the uppermost layers of skin (epidermis, dermis). This produces a slight thermal erythema, so that the gel (salve) with the active ingredient can be introduced more quickly and deeply into the tissue. The thermal erythema, a slight reddening of the skin, regresses around 10 minutes after the light exposure.

The measured pressure of the cap against the tissue being treated furthermore produces there a reduced blood flow, which results in a deeper penetration of the radiation, because blood is a strong absorber of light. This enables an optical treatment of deeper tissue layers with or without substances containing active ingredients.

Moreover, instead of or in addition to the substances containing active ingredients, certain fluids can also be applied to the cap as lubricating and/or immersion agents, in order to prevent annoying friction between the cap material and the skin during the treatment and/or to achieve an improved optical focusing of the light in the tissue.

### BRIEF DESCRIPTION OF THE DRAWINGS

In what follows, the irradiation appliance of the invention shall be explained more closely by means of FIGS. 1 to 6 and by means of specific sample embodiments. There are shown here:

FIG. 1, an exploded drawing of the treatment appliance according to the first sample embodiment of the invention;

FIGS. 2a and 2b, cross sectional views of the treatment appliance according to the first sample embodiment to explain the use according to the invention;

FIGS. 3a and 3b, further cross sectional views of the treatment appliance according to the first sample embodiment to explain the use according to the invention;

FIG. 4a, a perspective view of the treatment appliance according to the first sample embodiment of the invention;

FIG. 4b, a cross sectional view of the treatment appliance according to the first sample embodiment of the invention;

FIG. 4c, a cross sectional view of the tissue pressure element of the treatment appliance according to a second sample embodiment of the invention;

FIG. 5a, a perspective view of the treatment appliance according to a third sample embodiment of the invention;

FIG. 5b, a perspective view of the treatment appliance according to a fourth sample embodiment of the invention;

FIG. 6a, a cross sectional view of the treatment appliance according to a fifth sample embodiment of the invention;

FIG. 6b, a cross sectional view of the treatment appliance according to a sixth sample embodiment of the invention;

FIG. 6c, a cross sectional view of the treatment appliance according to a seventh sample embodiment of the invention;

FIG. 6d, a cross sectional view of the tissue pressure element of the treatment appliance according to an eighth sample embodiment of the invention; and

FIG. 6e, a perspective view of the tissue pressure element of the treatment appliance according to a ninth sample embodiment of the invention.

### DETAILED DESCRIPTION OF SEVERAL SAMPLE EMBODIMENTS

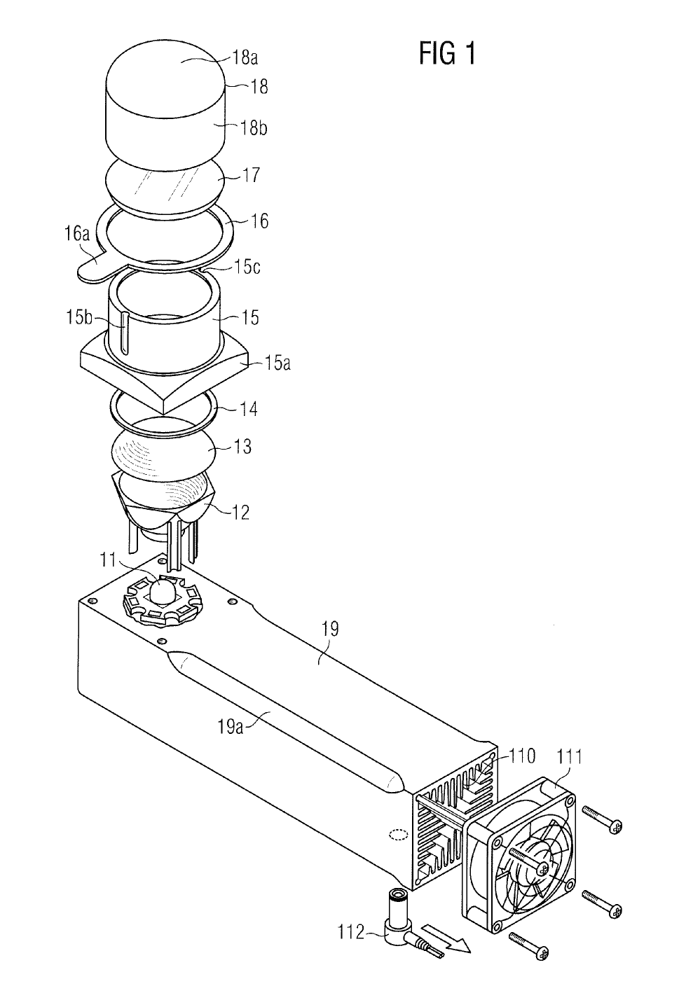

FIG. 1 shows schematically the layout of the dermatological irradiation appliance of the invention in the form of an exploded drawing.

The radiation source (11) preferably contains one or more light-emitting diodes (LEDs). Especially preferred is a diode array, which is assembled from 4 or 6 or even more LEDs, most of them connected in series. But in battery operation, the LEDs can also be hooked up in parallel by pairs. The total electric power of the diode array is between 5 and 25 W, preferably in the range of 5 to 20 W, especially preferably in the power range of 8 to 18 W.

The diodes of the array can emit in the spectrum interval between 320 nm and 150 nm, preferably between 350 nm and 1000 nm. The diodes can all emit in a uniform spectrum interval or be mixed in two different spectrum intervals.

Thus, for example, an array consisting of 4 single diodes can be used, 2 diodes emitting in the red region and 2 diodes in the blue region. One can also combine 2 diodes in the blue region and 2 diodes in the UVA region with each other. The choice of the spectral regions of the diodes depends on whether one wants to achieve a greater depth of penetration in the tissue (red, near infrared) or a lesser depth of penetration (blue, violet) or even a photochemical effect by radical formation (UVA, violet).

The diode array (11) is connected in good thermally conducting manner to an elongated cooling element (19) of aluminum with inwardly directed cooling fins (110), which also serves here as a handle for the manually operated irradiation appliance. The cooling element handle (19) has a length of around 120-140 mm and a rectangular cross section of 30×30 mm. The 4 lengthwise edges (19a) of the cooling element (19) are rounded so that one can hold it comfortably in the hand.

The fan (111) provides the necessary air flow for cooling of the diode array (11) and the handle (19), because the waste electric power can still amount to around 12-15 W.

The electrical power supply voltage, which lies in the medically safe low-voltage range (typically 15-24 V), is provided by the coaxial plug (112), and in the most simple case it is provided by a low-priced mains plug (typically 15 VDC, 1 A at most).

For efficient assembly, it is advantageous for some of the interior cooling fins (110) on the end of the handle (19) near the fan to be cut off (not shown here), so that the wiring of the diode array (11) to the bushing of the plug (112) and the accommodating of the required ballast resistors for the array and the fan and possibly other electronic components can be done conveniently.

The radiation which is emitted by the light-emitting diode (11) or the array of several such diodes first goes to a conical reflector (12) with highly reflective inner wall. The reflector (12) can also have parabolic symmetry, in which case the glass dome arranged above the array is situated approximately at the focal point of the reflector paraboloid.

Immediately after this come a lens (13) of glass of Plexiglas, which can be for example a planoconvex lens with a focal distance of 3-10 cm. Instead of a lens, one can also use an optical planar surface, especially when the reflector (12) is a parabolic reflector. Reflector (12) and lens (13) can also be omitted if the inner surface of the tube (15) is highly reflective.

Lens (13), reflector (12) and diode array (11) are situated inside a tube (15) with base plate (15a), which in this example are configured as a homogeneous aluminum part. The lens (13) can be mounted at both ends, once at the edge of the reflector (12) and at the other end by a projection not visible here inside the tube (15), while an O-ring (14) of an elastomeric material can be positioned between lens (13) and the projection in the interior of the tube (15), to protect the lens against mechanical vibrations.

In the outer circumferential surface of the tube (15) there is a groove (15b) running vertically downward, which starts at the upper margin of the tube (15) but does not necessarily extend to the level of the base (15a). It can end 1-2 mm before this. The groove (15b) is around 1-2 mm in width and only a few 1/10 mm deep. Another groove (15c) of the same geometry can be staggered by 180° and likewise located on the circumferential surface of the tube (15).

A metal ring (16) with lug (16a) is slipped over the tube (15) and mounted on the base (15a). The inner diameter of the ring (16) is several 1/10 mm greater than the outer diameter of the tube (15), so that the ring can be tilted somewhat by manual pressure on the lug (16a). The position of the lug (16a) is preferably directly underneath the groove (15b) or staggered by 180° to the groove (15b).

A cap (18) of highly transparent silicone rubber is slipped onto the tube (15) in most applications of the dermatological irradiation appliance, the envelope surface (18b) of the cap (18) being shoved until it makes contact with the base (15a) or with the ring (16). In this position of the cap (18), the groove (15b, 15c) should still by covered by at least 1 mm or so of the envelope surface (18b) of the cap (18). Since the silicone cap (18) sits rigidly on the tube (15), a thin layer of a grease or a highly viscous oil (silicone grease, silicone oil, vacuum grease, Vaseline, etc.) is applied on the outside of the tube (15), so that the cap (18) can be easily slipped along and exchanged, while when the groove(s) (15b, 15c) are covered by the cap (18) an airtight closure of the interior of the tube (15) is produced, consisting of the volume between lens (13) and light exit surface (18a) of the cap (18).

Provided that the lens (13) is also glued or press-fitted airtight inside the tube (15), one thus gets an airtight volume inside the tube (15) between lens (13) and the convex or slightly convex outwardly arched light exit surface (18a) of the cap (18). Assuming suitable dimensioning of the wall thickness and the inner diameter of the cap (18), as well as suitable values of the Shore A hardness and the elasticity values (stretchability) of the highly transparent addition-linked silicone rubber, one can easily transform the convex surface (18a) of the cap (18) into a concave inwardly arched surface (18a) of the cap (18) by pressing with the finger or thumb or by using a suitable shaped tool, and it will then stay like this due to the partial vacuum created in the space defined by the lens (13) and the light exit surface (18a) of the cap (18). This partial vacuum is caused, first, by the air being forced out from the volume beneath the cap surface (18a) and secondly by the restoring force of the deformed cap surface (18a).

If one now presses the lug (16a) of the metal ring slightly up (or down), so that the cap (18) on one side is lifted by around 2 mm, air gets into one of the grooves (15b or 15c) and the concave surface of the cap (18) is transformed back into a convex surface (18a).

The concave, inwardly arched surface (18a) of the cap (18) forms a trough, which is very well suited to holding the minimally required amount of a gel (cream, paste, liquid) containing an active ingredient, and the gel will be rubbed or massaged by circular motion and slight pressing of the cap (18) against the tissue. This occurs under simultaneous irradiation with the light of the LEDs. When the gel (cream, paste, liquid) has been spread onto the skin, the trough of the cap (18) can be transformed back into a convex (or planar) surface by pressing on the lug (16a) of the ring-shaped ventilating lever, which is better suited to the further spreading and rubbing in of the gel while at the same time irradiating the tissue with the LED light.

If the layer thickness d of the light exit surface (18a) of the cap (18) is not greater than 1-3 mm, and if the Shore A hardness of the addition-linked highly transparent silicone rubber is less than 90, preferably around 50, and finally if the slipped-on cap (18) still projects somewhat (e.g., 0.5-15 mm) beyond the edge of the tube (15) so that when the cap is pressed against the tissue one does not feel the hard edge of the metal tube (15), one obtains a soft flexible tissue pressure element that molds like a cushion to all irregularities of the tissue upon slight pressing, and thus ensures a uniform optical distribution and introduction of the gel (salve, paste, cream, liquid) with active ingredient into the tissue.

The plate (17) situated in the beam path serves as a wavelength shifter and can be used optimally, due to fluorescent substances contained therein, when one wants to generate a longer-wave radiation penetrating deeper into the tissue with the same irradiation appliance and the same LEDs (or LED array).

In order to reduce the glare of the LED radiation emerging from the pressure cap (18), which is especially troublesome in the wavelength region around 460 nm or in the longer wave spectral region, one can take the following steps: the LED should only shine when the pressure cap (18) has tissue contact, in which case a defined minimal pressure of the cap (18) against the tissue is transmitted to a microswitch, which in turn switches the LED current. This mechanism can be realized, e.g., by the tube (15) of FIG. 1 containing a second, thin-wall tube lying concentrically inside it, which can execute a minimal easy stroke motion of only a few millimeters inside the first outer tube (15) and which carries the tissue pressure cap (18) on its light exit opening.

The inner movable tube projects beyond the outer guide tube (15) by around 1-2 cm, so that the tissue pressure cap can be slipped onto it. The lower margin of the inner tube has mechanical contact with the trigger pin (lever) of a microswitch, which is situated on the handle piece (19) of the irradiation appliance of FIG. 1.

Thanks to the stroke movement of the inner tube upon tissue pressure of the cap, the switching mechanism of the microswitch is triggered and the LED shines. When the pressure cap is lifted off from the tissue, usually the restoring force of the microswitch is enough, but one can also strengthen it by additional use of a spiral spring to push up the movable inner tube, whereupon the LED light source goes out at once.

Alternatively, instead of the microswitch, one can also use a reed relay as the switch trigger for the LED light. The inner movable tube can then be provided with a permanent magnet (e.g., a ring-shaped permanent magnet) and an elastic restoring device, e.g., in the form of a spiral spring. Upon tissue pressure of the cap, the permanent magnet approaches the reed relay and at a minimal distance from this it brings about the contact switching (R≈0Ω) of the reed relay. When the cap (18) is lifted off from the tissue, the inner movable tube is again forced out by action of the spiral spring and the reed relay switches back to its starting state (R≈∞Ω), whereupon the LED light goes out.

The switching mechanism with the reed relay is somewhat more complicated than the layout with microswitch, but it needs no definite threshold value for the tissue pressure. The necessary stroke path of the inner tube for the triggering of the switching is longer, however.

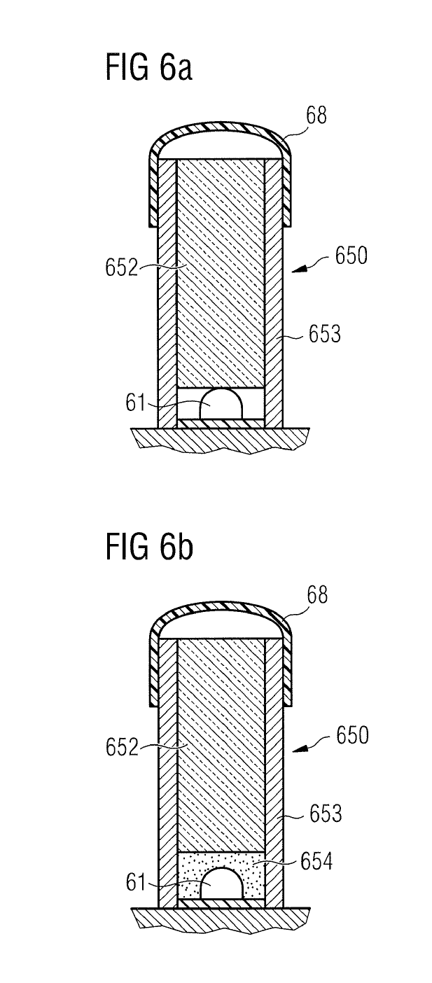

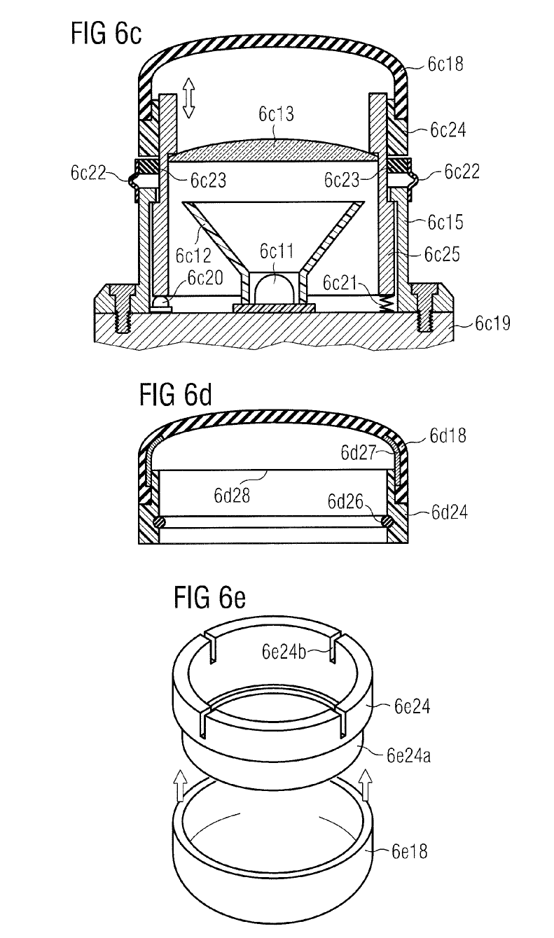

FIG. 6c illustrates the device which only shines upon tissue pressure. The pressure cap (6c18) including the base (6c24) is connected here firmly but interchangeably to an inner movable tube (6c25), which is mounted telescopically inside an outer tube (6c15) so that it can execute a small stroke movement (around 1-2 mm) inside the outer tube (6c15).

Both tubes (6c25) and (6c15) are intermeshed, so that the stroke movement is limited. Upon tissue pressure against the cap (6c18), the inner tube (6c25) presses against the microswitch (6c20) and optionally also on a spiral spring (6c21), by which the LED (6c11) is activated and shines. When the device is lifted off from the tissue, the restoring force of the microswitch (6c20) and the spiral spring (6c21) ensures that the inner tube (6c25) goes back to its starting position and the LED (6c11) goes out.

The silicone cap (6c18) is glued to a ring-shaped firm base (6c24), which ensures fast interchangeability of the tissue pressure element. Moreover, a ring-shaped end stop (6c23) is provided for the base (6c24). A seal of the gap between inner and outer tube, e.g., in the form of a smoothly moving (mono) bellows (6c22), prevents liquid substances from getting into the space of the LED (6c11), with reflector (6c12) and lens (6c13), both of which can also be omitted if the inner tube has a highly reflective inner wall. Alternatively, a light guide rod similar to FIG. 6a can be mounted on the inner movable tube, whose light exit surface is placed against the tissue, and the LED only shines when the light guide rod has tissue contact.

A further reduction of the glare of scattered light emerging from the highly transparent silicone cap (18) and especially from the cylindrical circumferential surface (18b) can be accomplished by coloring the cap.

One can color the cap so that the cylindrical circumferential surface (18b) is light absorbing and only the frontal light exit surface (18a) remains highly transparent. It is also enough to make only the region of the cylindrical envelope surface of the cap light absorbing, directly adjoining the transparent light exit surface (18a) of the cap (18).

This can be achieved, e.g., by inserting a cylindrically shaped black or light absorbing colored hose segment into the mold for the cap prior to filling the mold, so that it is integrated in the final solidified cap. Such a hose segment consists, e.g., of a cut-off ring from a thin-wall black-colored silicone hose, which can be around 10 mm in height and inserted so that it borders directly on the frontal light exit surface of the cap.

If one combines the features of light shutoff when no tissue pressure with the light-absorbing attribute of the cap (18) on at least part of its cylinder surface (18b), one can do without the use of protective glasses usually when massaging salves or gels with active ingredients into the tissue by means of intense visible LED light, which constitutes a significant simplification of the method.

FIGS. 6d and 6e illustrate the overall removable and interchangeable tissue pressure element consisting of silicone cap (6d18) and the ring-shaped base (6d24, 6e24) of plastic or metal, on which the cap (6d18) is glued. The silicone cap (6d18) has a glare protection (6d27) on its inner cylindrical wall, which projects beyond the edge of the fixed base (6d24) at least in the region and goes as far as the margin zone of the convex light exit surface.

If the silicone cap (6d18) projects a few millimeters (such as 5-15 mm) more beyond the firm edge (6d28) of the base, one achieves an optimal conformability of the light exit surface of the cap (6d18) to the irregularities of the tissue surface, which is very desirable primarily for applications in beauty care (wrinkle treatment) but also in medicine for pain treatment by irradiation of joints with tissue contact, possibly also with simultaneous introducing of painkilling gels or salves.

For this, it is advantageous that the silicone cap (6d18) is thin-walled (d around 1 to 3 mm) and the Shore A hardness is <90, preferably between 30 and 70. The glare protection (6d27) built into the cylindrical wall of the cap (6d18) can be, e.g., a thin-walled black band, which is glued onto the cap (6d18) from the outside or the inside or built in when casting the cap. It is also possible to bring about light absorption in the cylindrical region of the cap (6d18) by coating with black-colored silicone on the inside (6d27) and/or outside, e.g., by “dip coating”. The O-ring (6d28) in the base (6d24) ensures a good seating and sealing when the tissue pressure element is placed on the outer tube (15 FIG. 1) or on the movable tube (6c25) of FIG. 6c.

FIG. 6e shows yet again the assembly of base (6e24) with attachment (6e24a) for the cap (6e18). Instead of an O-ring, four slots (6e24b) on the underside of the base (6e24) provide for a firm clamping seat.

The use of the base (6c24, 6d25, 6e24), on which the pressure cap (6c18, 6d18, 6e18) or the light guide rod (650) is secured and which is then mounted on the intermediate piece (6c15, 6c22, 6c23, 6c25) is shown only in FIGS. 6c to 6e. Even so, this important element of the present invention for improving the easy removability and interchangeability of the tissue pressure element is not limited to this specific sample embodiment, but rather can and should also be used preferably in the other sample embodiments. In particular, a base element (not shown) can be arranged between the intermediate piece (15; 25; 35; 45; 55; 550, 551; 650) and the attachment element (18; 28; 38; 414; 418; 58; 68) in FIG. 1-4b and 5b-6b.

The description of preferred sample embodiments of the invention will now be continued by means of FIG. 1. The plate (17) can consist, e.g., of Plexiglas or another plastic, which is doped with a perylene colorant, especially perylene orange (yellow) or perylene red or, in the customary designation, Lumogen® orange (yellow) or Lumogen® red. If one uses the LED (11) or the LED array (11) in the blue spectrum region, e.g., in the region of 450 nm≦λ≦480 nm, this radiation lies optimally in the fluorescence excitation spectrum of the two colorants Lumogen® red or Lumogen® orange (yellow). But in this case one generates highly divergent fluorescent radiation in both the forward and backward direction.

If one presses the plate (17), which can be coated only with the thin highly transparent silicone layer of the cap (18), against the tissue, e.g., skin tissue, at least the highly divergent (red, yellow) fluorescent radiation that emerges from the plate (17) in the forward direction will be absorbed in the tissue. The fact that the fluorescent radiation emerges highly divergent from the plate (17) is not so much of a disadvantage, since the tissue itself constitutes an optically strongly scattering and absorbing medium. The fluorescent plate (17) can also consist of glass, which is coated with other fluorescing colorants, such as colorants based on zinc sulfide or oxides of rare earth metals.

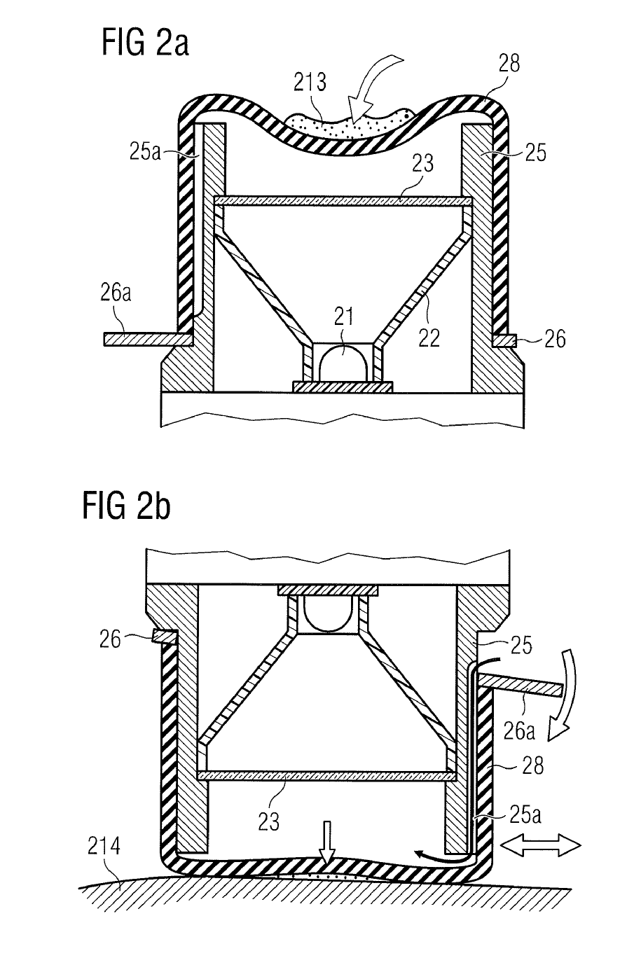

FIGS. 2a and 2b show in detail the mechanism of changing the geometry (convex/concave) of the tissue pressure surface of the highly transparent silicone cap (28).

Before adding the gel (cream, paste, salve, liquid) (213) containing the active ingredient, the cap (28) is pressed down, preferably by hand, to produce the trough shown in FIG. 2a. This trough remains in place, since the pressing down forces most of the air out from the space beneath the trough as far as the optical plate or lens (23), creating a partial vacuum in this volume. The prerequisite is that the optical plate (lens) (23) be mounted airtight in the tube (25). The mounting is done preferably by gluing and/or an O-ring seal.

Next, the minimally required quantity of substance (213) with active ingredient to be introduced into the skin is added to the trough, for example, a gel containing Hyaluron®. After this, the substance is distributed over the area of tissue by circular motion with slight pressure of the cap (28) on the tissue, with or without light. Once the substance (213) is distributed over the tissue, the surface of the cap (28) is returned to its slightly convex or at least planar shape by pressing on the lug (26a) of the ring lever (26), since the lever (26, 26a) shoves the margin of the cap (28) far enough so that air can get into the groove (25a), whereupon the pressure surface of the cap (28) goes back to its original shape. Of course, the elastic restoring force of the deformed cap helps out in this process.

The further and actual introducing of the substance with active ingredient into the tissue then occurs by pressing the now convex (planar) cap surface against the tissue (214) with simultaneous slow circular motion and irradiation by light until a dry state of the tissue surface is achieved.

With the help of this technique, one can make do with a minimal amount of the substance (213), since it does not come into contact with the hands of the care provider and no substance can seep into the silicone material. This sparing use of gel (salve, cream, paste, liquid) with active ingredient is important, because such substances (213) are extremely expensive, especially in the case of substances containing Hyaluron®, and they play a major role in the invention being specified.

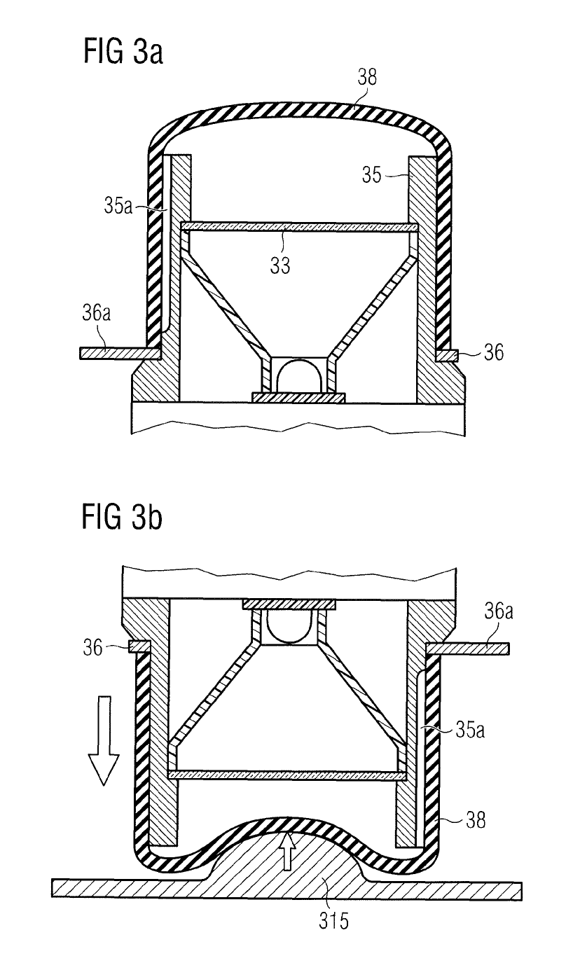

FIGS. 3a and 3b show how one can use a simple tool (315) in the shape of a flat plate with a convex eminence to press the trough into the cap (38). In this process, air escapes outside from the compressed volume between the inner wall of the cap (38) and the outer wall of the tube (35). The prerequisite is that the glass plate (lens) (33) be mounted airtight inside the tube (35). Of course, the trough can also be forced manually into the cap (38) by finger or thumb pressure without a tool.

By pressing on the lug (36a) of the ring-shaped ventilation lever (36), the trough can be removed once more as soon as the raised margin of the cap (38) releases the ventilation through the groove (35a). Usually a displacing of the cap margin that covers the groove by only around 2 mm is enough for this. In the sample embodiment of FIG. 3a, the lug (36a) of the ventilation ring (36) is situated directly next to the emerging ventilation groove (35a). But it is just as possible for the lug (36a) and the groove (35a) to be staggered by 180° from each other. In the former case, the lug (36a) has to be moved upward from below for the ventilation and in the latter case in the opposite direction. Both options are equivalent.

The cap in this sample embodiment has an inner diameter of 26 mm, a homogeneous wall thickness of 1-2 mm, both in the cylindrical part and in the slightly convex frontal region, where it can also be somewhat thicker or thinner in any case, e.g., 2 or 2.5 mm or less than 1 mm. The length of the cap (38) is around 23 mm. The Shore A hardness of the highly transparent addition-linked silicone rubber used here and preferred is preferably in the range of 20-90, in this example, 45 (ISO 868). The relative elastic stretchability to breaking is around 100% (ISO 37).

The material and geometry of the cap are chosen so that its pressure surface when pressing against a flat hard test surface (with a pressure of at least 0.5 N/cm²) is increased by at least 5%, preferably around 10%.

The cap is produced by mixing the liquid components A and B, here in a ratio of 9:1, then making it free of bubbles in a vacuum chamber and pouring into a corresponding mold, in which the cross-linking takes place at elevated temperature. One can very easily shove the cap (38) onto the metal tube (35) and remove it again if the tube (35) is provided on the outside with a thin layer of a highly viscous oil (silicone oil) or grease (Vaseline), which only needs to be applied once or seldom.

The material of which the cap (38) consists is highly transparent, so that around 90% optical transmission can be measured in the range of 350 to 980 nm with a layer thickness of 1 to 2 mm. This is important so that none of the radiant energy is “given away”.

Furthermore, the material is tissue compatible, it can be disinfected with all commonly used medical products, and moreover it can be autoclaved. The material used here can even obtain medical certification per ISO 10993-1 and USP Class VI. Its good chemical inertness is likewise important, because in the present invention it comes into contact with many different liquid or creamlike or pastelike vehicle substances and active ingredients.

One can also color the pressure cap (18, 28, 38) of silicone (or another transparent material) overall with a light absorbing coloring material. In the extreme case, the coloring material contains finely divided graphite powder and the silicone cap becomes totally black. For this, it is enough to add the black coloring material containing graphite in only one percent by weight. Such a black silicone cap is quickly warmed up by the LED radiation impinging on the inside and can likewise be used to work in salves (gels) with active ingredients thanks to the heat of contact that is transmitted to the tissue. Of course, there is no glare present.

But the working principle of the black cap is totally different as opposed to a heating of the tissue by radiation absorbed in the tissue when using a transparent cap: the black, heated cap gives up its heat to the tissue by the slow process of thermal conduction, as opposed to the instantaneous heating by radiation absorbed in the tissue, which also acts at once within the depth of penetration. Yet one benefit of the black cap is that it quickly warms up a substance (213) with active ingredient that is applied to the surface of the cap, with the result that its viscosity is decreased, and it can penetrate more quickly and deeply into the tissue. Most salves and gels are heated only slowly if at all by direct radiation absorption, because they absorb little if at all in the visible and near infrared spectral region (360≦λ≦950 nm).

In this context, a gray cap is also of interest, being colored with graphite-containing coloring agents so that the front light exit surface still lets through incident LED light, such as 10% to 90%, preferably 20% to 80%, and the other portion of the radiation is absorbed in the light exit surface (18a) of the cap (18) and heats it. Thus, one has available a combination of the two mechanisms for introducing substances with active ingredients in tissue, depending on the choice of the optical transmission of the light exit surface of the cap (18), which can also be quickly exchanged for another one with different optical transmission.

The glare of the “gray” cap is at least greatly reduced, especially for transmission levels of T≦60%, which is also advantageous. The glare of the “gray” cap can also be reduced if the wall thickness of the cap in the cylindrical part is greater than in the frontal region.

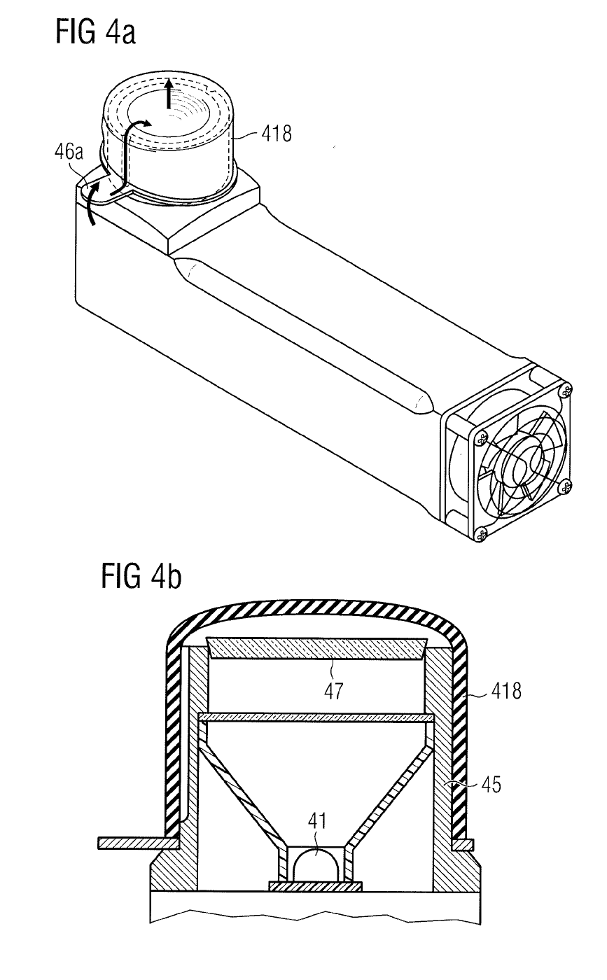

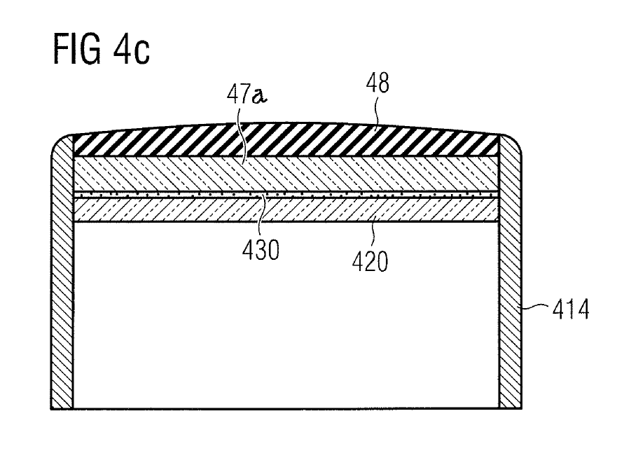

FIG. 4a shows the dermatological treatment appliance fully assembled with cap (418) in place and slightly raised ventilation lug (46a), whereby air from the outside can get through the ventilation groove into the tube, changing the concave shape of the light exit surface of the cap (418) back into a convex (or planar) one.

FIG. 4b shows the mounting of the fluorescent plate (47) inside the tube (45). The fluorescent plate, preferably a Plexiglas disk doped with one of the perylene dyes Lumogen® red or Lumogen® yellow or Lumogen® orange, has a loose fit inside the tube (45), because it is supposed to be interchangeable. The silicone rubber cap (418) encircles the tube (45) here, including the fluorescent plate (47).

If one chooses a diode array (41) with 4 single diodes, all of them emitting uniformly in the blue spectral region between 455 nm and 475 nm, this radiation will be entirely absorbed in the 3 mm thick plate (47), which is highly doped with Lumogen® red and/or yellow or orange, and transformed into longer wave fluorescent radiation having its spectral maximum at roughly 580 nm or 630 nm and emitted by the dye molecules in the entire solid angle of 4π. One half of the fluorescent radiation will be emitted in the forward direction and the other half in the backward direction, back to the diode (41).

But one can again deflect to the front the fluorescent radiation scattered in the backward direction by placing a glass plate of around 1 mm thickness and the same diameter as the plate (47) in front of the plate (47), i.e., at the diode side, in sandwich fashion, and having a short-pass thin-film filter vapor deposited onto it. This filter has the property that the pumping or exciting radiation in the interval of 455 nm<λ<475 nm is almost 100% transmitted, and the fluorescent radiation in the interval of 550 nm<λ<730 nm is almost 100% reflected. The edge length of the short-pass filter is in the interval of 480 nm<λ<510 nm.

In this way, the portion of the fluorescent radiation emitted to the rear, in the direction of the diodes (41), can be deflected to the front in the direction of the radiation exit opening, so that one can measure an intensity boosting of the useful fluorescent radiation of a good 35%. Unfortunately, owing to the natural absorption of the fluorescent radiation in the material highly doped with the perylene dye, a 100% boosting is not possible.

Thus, with the help of the fluorescence technique described here, at a pumping power in the blue of around 2.5 W, one can achieve a radiant output power in the yellow-red spectral region of around 700 mW from a light exit opening of around 25 mm diameter. This radiation power density of around 140 mW/cm² is already enough to produce a perceptible feeling of warmth on the skin. The radiation in the longer wave yellow and red spectral region penetrates much more deeply into tissue than the radiation in the blue region and therefore it can be used for medical or cosmetic purposes.

FIG. 4c shows an alternative arrangement of the fluorescent plate (47) to FIG. 4b, making it possible to switch quickly from blue to yellow-red or red or white light radiation with simple means. The fluorescent plate (47a) in FIG. 4c is located in an insertion sleeve (414) of metal or plastic, which is placed on the tube (45) of FIG. 4b instead of the cap (418). The radiation exit surface of the fluorescent plate (47a), as shown in FIG. 4c, can be coated with a thin, highly transparent or at least translucent layer (48) of plastic, preferably silicone rubber once again.

On the opposite surface of the fluorescent plate (47a), a thin-film filter (420) can adjoin it, congruent with the plate (47a), and having the already described short-pass property, i.e., greatest possible transmission in the blue region and greatest possible reflection in the yellow-red region of the spectrum. The short-pass filter (420) can be vapor deposited on a glass plate of around 1 mm thickness, which can be joined across a thin layer (430) of an optical adhesive, such as an acrylate adhesive, to the fluorescent plate (47a) in sandwich fashion. The sandwich, consisting of the two plates (420 and 47a) is glued in the margin region to the ring-shaped insertion sleeve (414), which in turn can be mounted with a clamping fit on the tube (45), FIG. 4b.

The short-pass filter in thin-film technology can also be applied directly to the perylene-doped Plexiglas plate (47a), e.g., by the sol gel technique. In any case, the short-pass filters with optimal optical properties are vapor deposited on glass substrates.

It may be advisable for the short-pass filter (420) to not be firmly connected to the wavelength shifter, whether in the form of a fluorescent plate (17) or in the form of a cap (18) doped with fluorescent dyes. In this case, the short-pass filter can be beneficial, even with pure blue light irradiation, because it cuts out disruptive longer wave background radiation of the LEDs and therefore enables a more contrast-rich fluorescent diagnosis (e.g., for nail fungus) by means of a long-pass filter. This is also beneficial when the irradiation appliance is used as a forensic lamp to search for traces of blood, sperm or saliva.

The fluorescence technique described here makes it possible to use a single LED light source in simple and economical manner in four different color ranges (blue, yellow, red, white) with different depth of penetration in tissue for medical or cosmetic applications or only for illumination. The alternative would be to use four different LED radiation sources, which is also possible, of course, but more costly. The fluorescent technique is not limited to the use of blue light as pumping radiation. But it works especially well here, due to the maximum efficiency of the diodes in the blue region and the fact that the especially efficient perylene fluorescent dyes Lumogen® red and Lumogen® yellow (or Lumogen® orange) have their strongest absorption and most effective pumping band in the blue.

One can adjust the thickness of the fluorescent plate (47, 47a) so that the blue pumping radiation is transformed entirely into longer wave fluorescent radiation. Such a plate then has, say, a thickness of 3 mm. If one chooses a lesser plate thickness of, say, only 2 mm or 1 mm, increasingly more blue pumping radiation will go through the plate (47, 47a), so that one gets mixed light, consisting of long and short-wave fractions. Such a mixed radiation can also be useful in dermatology or beauty care for smoothing of wrinkles or other applications (e.g., acne), because it produces a less sharp temperature profile in the skin, i.e., a somewhat lower surface temperature, but also a greater depth of penetration down to the subcutis.

Thus, for example, an irradiation with blue light (455 nm≦λ≦475 nm) as well as with red light or yellow-red light in the range of 560 nm≦λ≦780 nm has proven to work well for wrinkle smoothing with the help of substances containing Hyaluron® (gel, liquid). It is also possible to incorporate fluorescent dyes directly in the silicone rubber cap (18, 28, 38, 418) making contact with the tissue in order to transform the LED light into longer wave fluorescent light or at least obtain a longer-wave color mixture in with the blue LED light, for example, to achieve a greater depth of penetration of the radiation in the tissue.

Suitable for this are the Lumogen® dyes from the perylene group, which are well soluble in the nonpolar silicone oils and therefore can also be easily incorporated in the silicone rubber cap. Yet other fluorescent dyes can also be incorporated in the liquid phase of the silicone prior to the molding and cross-linking of the cap.

Thus, in order to produce longer-wave fluorescent light in the yellow and red spectral region when excited by blue light, it is possible to incorporate, besides Lumogen® dyes, also the following fluorescent dyes or phosphors in the material of the tissue pressure cap made of silicone, but which can also consist of another transparent polymer or elastomer or rubberlike material: phosphors based on rare earths, such as cerium, samarium, europium, terbium, neodymium and others, which in turn are usually embedded in a glass matrix, such as (Sr, Ba, Ca)₂ SiO₂ or in a crystalline matrix such as yttrium aluminum garnet (Y₂Al₅O₁₂), and are available in finely pulverized form.

But also dyes or phosphors based on transitional metals such as Ti, Cr, Mn, Fe, Co, Ni, Cu and others, when incorporated in a crystalline matrix and present in pulverized form, can be incorporated in the tissue pressure cap or mixed in prior to the final cross-linking (silicone) or hardening.

As an example, one can mention a phosphor consisting of pulverized ruby, i.e., chromium ions in a matrix of crystalline Al₂O₃. This phosphor absorbs in the blue and violet spectral region and fluoresces in the long-wave red spectral region at 694 nm.

Or one incorporates in the silicone of the tissue pressure cap finely pulverized Plexiglas (acrylic glass) or other pulverized transparent plastics that have been doped with a perylene (Lumogen®) dye, so that the perylene molecules are present in a matrix of acrylic glass or another plastic, which is beneficial, because the perylenes in a matrix such as acrylic glass are especially photostable and fluoresce especially effectively.

Furthermore, one can also incorporate in the pressure cap fluorescing phosphors based on quantum dots, such as CaTe, or (Cd, Se) ZnS or PbSe, also as nanopigments.

The phosphor-doped tissue pressure caps of silicone can also be coated, after the cross-linking, especially on the tissue pressure surface, with a thin layer of silicone (layer thickness≈0.1 mm to 1 mm), to prevent the phosphors from coming into contact with the tissue.

One can also mix in fine-grained SiO₂ powder with all these dyes or phosphors in silicone or other transparent polymers or elastomers to improve the homogeneity and the effectiveness of the emitted fluorescent radiation. The SiO₂ powder can be used here down to the finest possible grain size in the nano region.

Thus, with a silicone cap that is doped, e.g., with the rare earth dyes Eu and/or Ce, and with an addition of SiO₂ powder, one can produce very homogeneous diffuse white light when excited by a LED that emits in the blue region at around 460 nm, even being exceptionally suitable as diagnostic light, and this not only in dermatology applications, but also in forensics, for example.

SiO₂ powder by itself in the silicone cap, without the addition of another phosphor, acts as a light diffuser and can be useful in special cases of application of light, e.g., in body cavities.

For sake of completeness, it should be mentioned that one can also do without the cap deformation, as described for FIG. 2-3, especially when using less high-quality and costly gels (salves, pastes), or when using caps with larger diameter, because these can be more easily filled with gels (pastes, salves). Such larger caps have a diameter of the tissue pressure surface of >10 mm, such as 40 mm.

The pressure caps are preferably firmly joined to a ring-shaped substructure or base element of plastic (such as a duroplastic like Delrin®) or metal, which is turn can be mounted on the tube (15) of FIG. 1 with a clamping fit, so that the different caps of different size and with different spectral properties can be quickly interchanged for each other. Also see FIGS. 6d and 6e for this. Light guide rods including the optically insulating sheath, according to FIG. 6a, can also be mounted on such a base element (6d24), so that a quick changing of caps with slight radiant power density to small-surface applications with high radiant power density is possible.

Blue light only penetrates to the dermis, while longer-wave light of λ>500 nm also penetrates to the subcutis. Even so, light-emitting diodes emitting blue light have around three times higher radiant power for the same electric power (e.g., 10 to 15 W), due to the maximum optical efficiency of the LEDs in the blue. For this reason, one can achieve the effect of introducing the active ingredient into the uppermost layers of skin most quickly with intense blue light in the power density range of around 100 to 1000 mW/cm².

The constantly sliding motion of the radiation source on the skin surface is necessary for this high radiant power density, because otherwise the tissue would become too hot after around 5 to 10 seconds of static irradiation. But just such a perceptible heat production in the intense blue range of the spectrum is what stimulates the microcirculation in the uppermost skin layer (down to around 1 mm depth) and thus speeds up or improves the introducing of the active ingredient in the tissue.

The slight depth of penetration of the blue light as compared to red light also has the benefit that, e.g. in wrinkle treatment, one can work more closely in the region of the eye, without the eye being irritated by stray light.

The obvious requirement for the application of such intense light sources is of course the wearing of protective glasses, both for the patient and for the care provider. The wearing of sunglasses by the patient during the treatment has also proven to be good.

Instead of the fluorescent technique with perylene dyes or other, even inorganic dye molecules, one can also use the invented dermatological treatment appliance with a diode array with emission in the red or near infrared spectral region, e.g., in the region of 600 nm<λ<980 nm. Thus, a diode array with around 10 W electric power and a radiant power of around 1 W at a peak wavelength of 740 nm has proven to work well for wrinkle treatment with light.

This radiation has the benefit of lying in the spectral region of maximum penetration depth in tissue, yet still being visible, and not so glaring as shorter-wave red light (e.g., at 630 nm), which also penetrates less deeply. Longer-wave radiation in the near infrared region also does not penetrate more deeply into tissue (than 740 nm), but it is invisible and thus hazardous, especially for applications in the facial region. One can go ahead and use high-power diode arrays with emission at, say, 850 nm or 940 nm in areas outside of the face. But in any case, protective glasses are required.

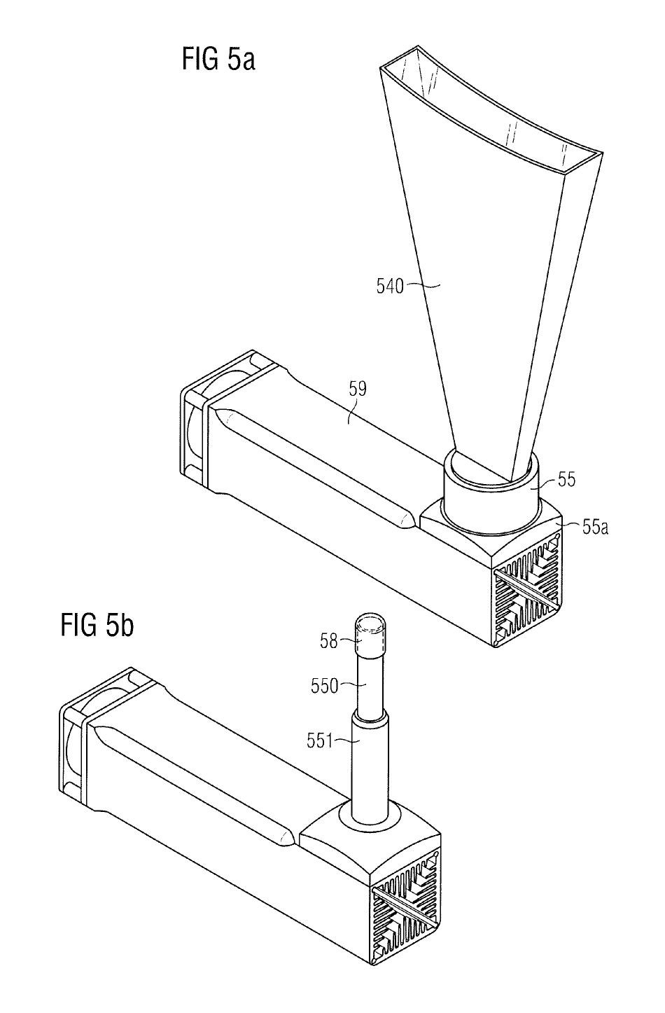

FIGS. 5a and 5b show variants of the invented irradiation appliance which use the basic unit, consisting of the handle (59) with fan and light-emitting diode array, represented in principle in FIG. 1. They differ only by special applicator attachments (540 and 541), making possible special beam shaping of the radiation emitted by the diode array.

FIG. 5a contains, as the applicator attachment, a cross section transformer, which changes the circular symmetrical emission radiation of the diode array into an oblong rectangular beam profile, such as can be used in dentistry for the bleaching of teeth in the “smile region”.

The beam transformer (540), also called a beam cross section transformer, consists here of a frustum-shaped hollow body with highly-polished reflective interior, fitted into the tube (55) with base (55a). The smaller base surface of the frustum, which is open, forms the beam entry window for the diode radiation, while the larger base surface, forming the beam exit window, is slightly curved and covered with a thin-wall transparent exit window (not shown here), so that one can adjust the cross section transformer up to a few centimeters distance from the region of the front and side teeth (the “smile region”) of the patient. The radiation emitted by the cross section transformer, in the form of a rectangular strip, overlaps the “smile region” of the patient.

If one uses a single diode array as the radiation source, consisting of 4 single diodes with peak emission at around 395 nm and an electric input power of around 15 W, one has a radiant power density of around 100 mW/cm² available for the bleaching on the tooth surface, which is quite sufficient for the efficient radical formation in the usual peroxide-containing pastes that are used for bleaching.

One can also use diode arrays with emission at 385 nm, or 360 nm, or even 455-470 nm for the same purpose. One can very easily produce the cross section transformer that is used here for the bleaching, as I have described in my earlier patent application DE 10 2010 050 962.0, so that the entire bleaching lamp of FIG. 5a is very cheap to produce on the whole, and moreover it can also be kept light and small in size. The disclosure as to the making and using of the cross section transformer in patent application DE 10 2010 050 962.0, especially as regards its FIGS. 12 to 14, is hereby taken up in its full extent by reference in the present application.

FIG. 5b and FIGS. 6a and 6b show the coupling of a rigid or flexible light guide (550) to the LED or the LED array, which is in thermal contact on the handle with cooling. The rigid or flexible light guide (550) is supported by a tubular shoulder with base (551). The light entry surface of the light guide (550) or (650) is situated practically in contact with the glass dome (61) of the LED array.

The light guide in this example is a quartz glass rod, which has a polished cylinder surface and likewise polished end surfaces. The light guide rod (652), which can consist of quartz glass or Plexiglas, for example, is optically isolated by a closely fitting Teflon® FEP or Teflon® MFA hose (653), which can have on its inner surface a Teflon® AF (amorphous fluoropolymer) coating.

In addition, yet another thin layer of a highly viscous perfluorinated liquid, such as a perfluoropolyether, can be situated between the inner wall of the isolating hose (653) and the cylinder surface of the light guide rod (652), acting as an optical immersion liquid due to its extremely low index of refraction in the range of n≈1.28 to 1.32 for the solid Teflon® AF layer, which likewise has an extremely low index of refraction in this range. This provides a very high optical aperture (2α˜83° in the case of quartz glass, and 2α˜93° in the case of Plexiglas) for the light guide rod (652), so that an especially high solid angle fraction of radiation of the highly divergently emitting diode array can be captured by the light guide and routed to the outside.

This described type of optical isolation of a light guide rod made of glass or plastic even allows a certain flexibility of the light guide rod (652) in the case of Plexiglas or another transparent plastic as the material for the light guide rod, without danger of fragments or sharp edges in event of breakage.

Thus, a light guide rod (652), which consists of Plexiglas, e.g., and has a length of 1000 mm and a diameter of up to around 6 mm, being optically encased by a thin-wall (d=0.5 mm) Teflon® FEP sheath, which has a layer containing Teflon® AF or Hyflon® AD on its inner surface, and which can furthermore contain a highly viscous perfluorinated liquid, can be bent by as much as 90° without noteworthy loss of transmission. If such a rather long light guide were to break, the envelope sheath of perfluorinated plastic surrounding it, such as Teflon® FEP or Teflon® MFA, which can have a wall thickness of 0.3 to 1.0 mm, protects against fragments and injury from sharp fracture edges. Also in the event that the light guide rod consists of glass or quartz glass, flexibility is assured for rod diameter up to 2 mm.

But the optically and mechanically isolating envelope sheath of Teflon® FEP or MFA (653) of the rigid or semiflexible light guide (652) need not necessarily have a solid amorphous perfluorinated inner layer, which is Teflon® AF, for example. It is also enough to have only a thin layer of the perfluorinated highly viscous liquid between the circumferential surface of the light guide rod (652) and the inner surface of the envelope sheath (653).

This liquid can be, e.g., a perfluorinated highly viscous polyether, having a boiling point of over 200° C. and an extremely low refractive index in the range of around 1.28 to 1.32. Such liquids are available under the trade names Krytox® (Dupont) or Fomblin® (Ausimont). In this case, the perfluorinated liquid acts as an immersion medium for the perfluorinated envelope sheath (e.g., Teflon® FEP or MFA), which also has a low refractive index (n=1.34) but is not entirely transparent like Teflon® AF. For light guide lengths of up to 10-100 cm, this simplification of the optical isolation furthermore causes no significant decrease in the transmission of the light guide.

FIG. 6b shows yet another optical immersion layer (654), which optically connects the emitting glass dome of the LED array to the light entry surface of the light guide (652). The volume between the light entry surface of the light guide (652) and the LED, or the dome, is entirely filled with the material of the immersion (654).

The immersion layer (654) here can consist of a highly transparent soft elastic silicone gel or silicone rubber with slight Shore A hardness (Shore A<100) or of polymethylmethacrylate. Thanks to the immersion layer, the light coupling can be improved, since the material of the optical immersion layer is bounded by the light guide isolation sheath (653). Between the immersion layer (e.g., Teflon® AF) of the isolation sheath and the material of the immersion there is a big difference in terms of the size of the refractive index. Thus, a large part of the radiation of the LED that otherwise would have been lost can get into the light guide (652) by the light guide effect.

FIGS. 5b, 6a and 6b show a cap (58, 68) that is set on the light exit end of the light guide (650). It consists of the same material as described in FIGS. 1 to 4. It enables direct tissue contact with the light exit surface of the light guide arrangement and can easily be interchanged.

In one practical sample embodiment, a light guide rod of quartz glass of 10 cm length and 8 mm diameter, optically isolated by a closely fitting Teflon® FEP sheath coated with Teflon® AF with an intermediate immersion layer of Krytox® 16350, is coupled to a diode array consisting of 4 single diodes, emitting in the range of 460 nm.

The LED array has an electric power of 15 W, and the overall emitted radiation a power of around 3 W. The radiation output power measured at the light guide end is still 2.8 W, while a power density of 5.6 W/cm² results directly at the light exit surface of the light guide. With this power density, already several important dermatological indications (spider veins, age spots, warts, etc.) can be treated by the thermal effect of the radiation with tissue pressure, an application that is otherwise only possible with the laser.

The optical isolation of the envelope sheath (653) can contain up to three different fluorinated polymers. The light guide (652), for example, consists of a homogeneous rod of quartz glass (652), polished on all sides, with cylindrical symmetry. On its envelope surface there are three preferably close fitting isolation layers:

The first layer in direct contact with the envelope surface of the light guide (652) consists of a fluid or a liquid polymer, which is perfluorinated, highly viscous, and has an extremely high boiling point (Ts>200° C.). Perfluoropolyethers are such fluids.

As an example of such fluids, one can mention Krytox®, Fombline® and Galden®. The fluid Krytox® 16350 is suitable, for example.

This layer is adjoined by a thin layer (d˜1λ−3λ) of a solid amorphous perfluorinated polymer. Teflon® AF or Hyflon® AD or perfluoroalkyl vinyl ether with elevated copolymer fraction are possible materials.

The outer layer is a protective sheath whose inner surface is coated with the layer of amorphous perfluorinated polymer. The protective sheath preferably consists of a fluorocarbon polymer and has a wall thickness of around 1/10 to 10/10 mm.

Perfluorinated polymers like Teflon® FEP, Teflon® MFA, Teflon® PFA, Teflon® PTFE are especially preferred materials for the protective sheath. Yet partially fluorinated polymers such as the terpolymer Hostaflon® TFB can also be used as the material for the outer sheath, due to the better flexibility of these sheaths.

For cost considerations, one can also omit the solid amorphous inner coating, such as that of Teflon® AF, and only use the layer of the perfluorinated or partly fluorinated liquid polymer as the direct contact medium with the envelope surface of the light guide (652). This less expensive optical isolation is also somewhat less effective, but still quite good for light guide lengths up to 20 cm.

In general, the immersion layer with the immediately following isolation layer (amorphous layer or protective layer) should provide an at least approximate index matching.

The liquid isolation layer also has a great installation advantage. It is placed on the envelope surface of the light guide (652) before the cladding of the latter. The light guide can then be easily shoved into the closely fitting protective sheath (653). Owing to its high viscosity and owing to its high boiling point, the liquid layer can remain in the light guide structure permanently. This optical isolation technique is not only applicable for rigid light guide rods of quartz glass, glass, or transparent plastic, but also for flexible light guide filaments of quartz glass, glass, or optical fibers already optically isolated with slight aperture angle, such as those of quartz glass, or optical fibers of Plexiglas®.

Around the dome of the LED (61) it is also possible to arrange an inwardly mirrored tube (not shown), made from aluminum, for example. The tube encircles both the light entry region of the light guide (652) for a few millimeters length with the smallest possible gap between the interior lumen of the tube and the circumferential surface of the light guide, and the glass of plastic dome of the LED (61) as much as possible. Ideally, the tube extends to the bottom plate, the PCB board of the LED. The mirror treatment on the inner surface of the tube can be electrolytic, or vapor deposited, or produced by gluing a reflective film to the inner surface of the tube. In the arrangement just described, the tube brings about a roughly 25% greater light exit power from the light guide (650), which is especially desirable for the effective dermatological treatment, in particular, of acne, age spots, warts or spider veins.

The connection of a rigid light guide to the diode array in the highly efficient manner described here also enables the use of the LED radiation in body cavities (nose, ear, throat, etc.), because higher radiant power densities can be produced closer to the site of treatment. It is likewise possible to use the appliance in FIG. 5b, 6a, 6b in dentistry for the polymerization of plastic fillings or for industrial applications in the curing of light-hardening plastics based on epoxides, acrylates or silicone elastomers. Thus, the soft, adaptive cap (58) makes it possible to place the end of the light guide on the filling during the polymerization of a dental filling, even under pressure, while the highest radiant power density can be used and oxygen inhibition of the polymerization at the surface can be reduced by mechanical displacement of the oxygen of air.

One can also use the cap (58) of soft, highly transparent silicone rubber from FIG. 5b as a disposable cap, so that the light exit surface of the rigid light guide, which can also be a somewhat curved fiber rod at the light exit end, is constantly clean and has maximum transmissibility for the polymerization radiation.

The cap (58) in FIG. 5b or (68) in FIGS. 6a and 6b can also have the shape of a long stocking (also interchangeable as a disposable part), which in medical applications enables contact of the rigid light guide (550) (650) with the tissue, on account of its capability of being sterilized or autoclaved.

In applications of the irradiation appliance per FIGS. 1 to 6 for light or UV hardening, for example, of materials based on epoxy-acrylate or silicone elastomer, where the radiation exit surface can or should make contact with the plastic being hardened, it can also be advantageous to use a cap whose radiation exit surface consists of a fluorocarbon polymer, due to the better antiadhesion property. Especially advantageous for this are perfluorinated polymers like Teflon® FEP or Teflon® MFA. Thus, thin films (d=0.5 mm) made from this material transmit over 80%, both in the UVA and the visible spectral region, and they are also sterilizable and autoclavable for medical applications.

1. Appliance for dermatological or cosmetic treatment of a patient comprising:
a base body (19; 6c19), having a radiation source (11; 21; 41; 61; 6c11) for emission of light in a wavelength region between 320 nm and 1500 nm,
an intermediate piece (15; 25; 35; 45; 550; 551; 650; 6c15; 6c25), which is placed onto the base body (19; 6c19) in order to direct the light from the radiation source (11; 21; 41; 61; 6c11) toward the patient's body region (214) being treated, and
an attachment element (18; 28; 38; 414; 418; 58; 68; 6c18; 6d18; 6e18), arranged on the light exit end of the intermediate piece (15; 25; 35; 45; 55; 550; 551; 650; 6c15; 6c25), which is designed to be brought into direct contact with the body region (214) being treated, wherein
the attachment element (18; 28; 38; 414; 418; 58; 68; 6c18; 6d18; 6e18) contains an elastomer or a fluorinated thermoplastic material and is deformable when pressed against the body region (214) being treated.

2. The appliance according to claim 1, further comprising:
a base element (6c24; 6d24; 6e24) with cylindrical basic shape, which is detachably placed on a base of the intermediate piece (15; 25; 35; 45; 6c15; 6c25),
wherein a rigid or flexible light guide arrangement (550; 650) or the attachment element (6c18; 6d18; 6e18) is firmly joined to the base element (6c24; 6d24; 6e24).

3. The appliance according to claim 2, wherein the base element (6e24) has a first section for placing it with clamping fit on the base of the intermediate piece (15; 25; 35; 45; 6c15; 6c25) and a second section (6e24a) for fastening or gluing the light guide arrangement (550; 650) or the attachment element (6e18).

4. The appliance according to claim 3, wherein the clamping fit of the first section on the base of the intermediate piece (15; 25; 35; 45; 6c15; 6c25) is accomplished by an O-ring (6d26), or by an essentially axially extending slot (6e24b), or by the O-ring(6d26) and the essentially axially extending slot (6e24b).

5. The appliance according to claim 4, wherein the base element (6e24) is formed from a firm material, wherein the firm material is chosen from duroplastic or metal.

6. The appliance according to claim 1, wherein the attachment element (18; 28; 38; 414; 418; 58; 68; 6c18) in the form of a cap is placed over the light exit end of the intermediate piece (15; 25; 35; 45; 550; 551; 650; 6c25) and clamps an outer circumferential wall of the intermediate piece (15; 25; 35; 45; 550; 551; 650; 6c25).

7. The appliance according to claim 1, wherein the attachment element (18; 28; 38; 414; 418; 58; 68; 6c18; 6d18; 6e18) contains an elastomer, wherein the elastomer has a Shore A hardness of less than 100.

8. The appliance according to claim 1, wherein at least a light exit surface of the attachment element (18; 28; 38; 414; 418; 58; 68; 6c18; 6d18; 6e18) consists of a highly transparent material.

9. The appliance according to claim 8, wherein the highly transparent material is an addition linked silicone elastomer, wherein the attachment element (18; 28; 38; 414; 418; 58; 68; 6c18; 6d18; 6e18) increases its pressing surface (18a) by at least 5% when pressed against a flat hard test surface with a pressure of at least 0.5 N/cm².

10. The appliance according to claim 1, wherein the attachment element (18; 28; 38; 414; 418; 58; 68; 6c18; 6d18; 6e18) contains a light absorbing substance without fluorescent property.

11. The appliance according to claim 10, wherein the attachment element (18; 28; 38; 414; 418; 58; 68; 6c18; 6d18; 6e18) contains 0.01 to 3 wt. % of the light absorbing substance without fluorescent property, wherein the light absorbing substance is carbon or graphite.

12. The appliance according to claim 11, wherein the light absorbing substance without fluorescent property absorbs between 10% and almost 100% of the light emitted by the radiation source (11; 21; 41; 61; 6c11) and converts it into heat.

13. The appliance according to claim 8, wherein at least the light exit surface of the attachment element (18; 28; 38; 414; 418; 58; 68; 6c18; 6d18; 6e18) consists entirely of the elastomer or the fluorinated thermoplastic material.

14. The appliance according to claim 7, wherein the elastomer is chosen from one of the following materials: synthetic rubber, silicone rubber, addition-linked silicone rubber, polyurethane or polyethylene.

15. The appliance according to claim 1, wherein the attachment element (18; 28; 38; 414; 418; 58; 68; 6c18; 6d18; 6e18) contains a fluorinated thermoplastic material, wherein the fluorinated thermoplastic material is chosen from one of the following materials: Teflon® FEP, Teflon® MFA, Teflon® PTFE, and Hyflon® THV.

16. The appliance according to claim 1, wherein the attachment element (18; 28; 38; 414; 418; 58; 68; 6c18; 6d18; 6e18) has the shape of a cap, whose tissue pressing surface (18a) can vary between a convex and a concave configuration.

17. The appliance according to claim 1, wherein the attachment element (18; 28; 38; 414; 418; 58; 68; 6c18; 6d18; 6e18) has on the inside or outside of an envelope surface a thin layer (6d27) of a strongly light absorbing substance without fluorescence property.

18. The appliance according to claim 1, wherein the light absorbing substance with fluorescent property is chosen from a phosphor based on perylene phosphors, graphite colored silicone, and rare earths.

19. The appliance according to claim 18, wherein the attachment element (18; 28; 38; 414; 418; 58; 68; 6c18; 6d18; 6e18) doped with the light absorbing substance is coated with a thin layer of undoped silicone at least on its tissue pressing surface (18a).

20. The appliance according to claim 1, wherein a fluorescent plate (17; 47; 47a) is arranged between the radiation source (11; 41) and the attachment element (18; 414; 418), being doped with a perylene phosphor or a phosphor with rare earths.

21. The appliance according to claim 20, wherein the fluorescent plate (17; 47; 47a) is fastened at the light exit end of the intermediate piece (15; 45) or at the light entry surface of the attachment element (414), wherein the attached element is doped with a perylene phosphor chosen from Lumogen® yellow, Lumogen® orange, and Lumogen® red.

22. The appliance according to claim 20, wherein the fluorescent plate (17; 47; 47a) consists of acrylic glass doped with the phosphors.

23. The appliance according to claim 1, wherein an optical short-pass filter (420) is arranged between the radiation source (11; 21; 41; 61; 6c11) and the attachment element (18; 28; 38; 414; 418; 58; 68; 6c18; 6d18; 6e18).

24. The appliance according to claim 16, wherein the attachment element (18; 28; 38; 414; 418; 58; 68; 6c18; 6d18; 6e18) has the shape of a cap whose wall thickness is 0.5 to 2.5 mm.

25. The appliance according to claim 1, wherein tissue pressing surface (18a) projects by at least 5-15 mm beyond the front end of the intermediate piece (15; 25; 35; 45; 55; 550; 551; 650; 6c15; 6c22; 6c23; 6c25), or projects by at least 5-15 mm beyond the edge (6d28) of the base element (6c24; 6d24; 6e24), or projects by at least 5-15 mm beyond the front end of the intermediate piece (15; 25; 35; 45; 55; 550; 551; 650; 6c15; 6c22; 6c23; 6c25) and projects by at least 5-15 mm beyond the edge (6d28) of the base element (6c24; 6d24; 6e24).

26. The appliance according to claim 1, further comprising a mechanism (6c20, 6c21) designed to
interrupt or reduce the voltage supply of the radiation source (6c11) when the pressing force of the attachment element (6c18; 6d18; 6e18) against the body region (214) being treated lies below a predetermined threshold value, and
restore or increase the voltage supply of the radiation source (6c11) when the pressing force of the attachment element (6c18; 6d18; 6e18) against the body region (214) being treated lies above the predetermined threshold value.

27. The appliance according to claim 26, wherein a base of the intermediate piece (6c15; 6c22; 6c23; 6c25) has a connection body (6c15) fixedly fastened to the base body (6c19) and a connection body (6c25) movably mounted in relation to the fixed connection body (6c15).

28. The appliance according to claim 27, wherein the mechanism (6c20, 6c21) has a switch element (6c20), which is arranged between the movable connection body (6c25) and the base body (6c19) and designed to be switched by the movable connection body (6c25) at a pressing force of the attachment element (6c18; 6d18; 6e18) against the body region (214) being treated beyond the predetermined threshold value to a state wherein the radiation source (6c11) is connected to the voltage supply or the voltage supply of the radiation source (6c11) is increased.

29. The appliance according to claim 28, wherein
the mechanism (6c20, 6c21) has an elastic element (6c21), which is arranged between the movable connection body (6c25) and the base body (6c19) and designed to force the movable connection body (6c25) away from the base body (6c19) and thereby switch the switch element (6c20) to a state wherein it interrupts the electrical connection of the radiation source (6c11) to the voltage source when the pressing force of the attachment element (6c18; 6d18; 6e18) against the body region (214) being treated lies below the predetermined threshold value, wherein the elastic element (6c21) is a spring.

30. The appliance according to claim 1, wherein the radiation source (11; 21; 41; 61; 6c11) is an LED or an LED array, whose total electric power is between 5 and 25 W.

31. The appliance according to claim 30, wherein the radiation source (11; 21; 41; 61; 6c11) emits light in the wavelength region of 320 nm<λ<1500 nm.

32. The appliance according to claim 1, wherein the intermediate piece has a light guide arrangement (550; 650) with a rigid or flexible light guide (652), formed from glass, quartz glass or acrylic glass.

33. The appliance according to claim 32, wherein
the light guide arrangement (650) has an outer isolation sheath (653), enclosing the light guide (652), and
the isolation sheath (653), having an inner surface, contains Teflon® FEP or Teflon® MFA.

34. The appliance according to claim 33, wherein a layer of a highly viscous perfluorinated liquid is located between the inner wall of the isolation sheath (653) and the envelope surface of the light guide (652), wherein the inner surface is coated with an amorphous perfluorinated polymer.

35. The appliance according to claim 33, wherein the amorphous perfluorinated polymer is Teflon® AF or Ausimont® AD.

36. The appliance of claim 1, further comprising:
at least one light guide arrangement (550; 650) for interchangeable placement on a base of the intermediate piece (15; 25; 35; 45; 6c15; 6c25).

37. The appliance according to claim 36, further comprising:
wherein the attachment elements differ from each other in at least one of the following: different diameters, different transmission for light in the blue wavelength region, different amounts of a light absorbing substance with fluorescing property, so that they shine green, yellow, red, infrared or white when exposed to the light from the radiation source (11; 21; 41; 61; 6c11).

38. An irradiation appliance comprising:
a base body (19; 59; 6c19), having a radiation source (11; 21; 41; 61; 6c11) for emission of light in the wavelength region between 350 nm and 485 nm,
an intermediate piece (15; 25; 35; 45; 55; 550; 551; 650; 6c15; 6c25), which is placed on the base body (19; 59; 6c19) to direct the light of the radiation source (11; 21; 41; 61; 6c11) toward an object being investigated,
an optical short-pass filter (420) arranged at the light exit end of the intermediate piece (15; 25; 35; 45; 55; 550; 551; 650; 6c15; 6c25), and
an attachment element (18; 28; 38; 414; 418; 58; 68; 6c18; 6d18; 6e18) or plate (17; 47) doped with fluorescent dye arranged detachably at the light exit end of the intermediate piece (15; 25; 35; 45; 55; 550; 551; 650; 6c15; 6c25), being designed to generate diffuse white light from the light emitted by the radiation source (11; 21; 41; 61; 6c11) by fluorescence excitation.

39. A treatment appliance for dental bleaching of a patient, comprising:
a base body (59), having a radiation source for emission of light in a wavelength region between 350 nm and 1000 nm,
an intermediate piece (55), which is placed on the base body (59), which contains or encloses the radiation source, and
an attachment element (540) detachably secured to the intermediate piece (55), containing an interior mirrored hollow body in order to transform the essentially circular-symmetrical emission radiation of the radiation source into an oblong rectangular beam profile and thereby adapt the radiation cross section of the emitted light to the size of the body region being treated.

40. The appliance according to claim 38, wherein the radiation source has an LED light source whose total electric power is between 5 and 25 W.

41. The appliance according to claim 39, wherein the radiation source has an LED light source whose total electric power is between 5 and 25 W.
